(19) United States
(12) Patent Application Publication
(10) Pub. No.: US 2024/0033059 A1
(43) Pub. Date: Feb. 1, 2024

(54) PREPARING A TOOTH TO GENERATE A DIGITAL FILE

(71) Applicants: John Paul Stipek, SR., Wellington, FL (US); John Paul Stipek, JR., Royal Palm Beach, FL (US)

(72) Inventors: John Paul Stipek, SR., Wellington, FL (US); John Paul Stipek, JR., Royal Palm Beach, FL (US)

(21) Appl. No.: 17/878,669

(22) Filed: Aug. 1, 2022

(51) Int. Cl. A61C 13/00; A61C 13/08; A61C 9/00; A61C 5/77

(52) U.S. Cl. CPC A61C 13/0004; A61C 13/0006; A61C 13/08; A61C 9/008; A61C 5/77

(57) ABSTRACT

One embodiment of the apparatus and method is a computerized system (3000) comprising; a handpiece (106) integrating at least one sensor (110) and attaching a cutting tool (104); and at least one other sensor (110) adhered to an arch (112), the arch (112) having a tooth (108) to receive a crown. Another embodiment comprises the computerized system (3000) having a capacity to facilitate configuring of a prepared tooth to generate a digital file. Another embodiment comprises the computerized system (3000) having the capacity to produce an auditory, lighted, or vibration signal, alerting the dentist to increase or decrease the line-of-draw surface tapers (118) as needed to fit a crown. Another embodiment comprises the computerized system (3000) having the capacity to produce signals, alerting the dentist to move on to other surfaces of the prepared tooth as soon as the reduced surface (116) has been adequately reduced to fit a crown. Some of the digital workflow embodiments facilitate configuring of the prepared tooth that fits a premanufactured crown while other embodiments create the digital file that facilitates fabrication of a custom crown.

### BACKGROUND—PRIOR ART

The following is a tabulation of some prior art that presently appears relevant:

U.S. Patents

| Patent Number | Kind Code | Issue Date | Patentee |
|---|---|---|---|
| 10758317 | B2 | 2020 Sep. 1 | Yuan |
| RE48318 | E | 2020 Nov. 24 | Kwon |
| 11007035 | B2 | 2021 May 28 | Fares |
| 11185395 | B2 | 2021 Nov. 30 | Barak |
| 11007035 | B2 | 2021 May 18 | Fares |

U.S. Patent Applications

| Publication Nr. | Kind Code | Publ. Date | Applicant |
|---|---|---|---|
| 20200293699 | A1 | 2020 Sep. 17 | Fisker |
| 20200315754 | A1 | 2020 Oct. 8 | Ciriello |
| 20210169607 | A1 | 2021 Jun. 10 | Fisker |
| 20220008163 | A1 | 2022 Jan. 13 | Ozery |
| 20220183789 | A1 | 2022 Jun. 16 | Ciriello |

### FEDERALLY SPONSORED RESEARCH OR DEVELOPMENT

None.

### TECHNICAL FIELD

The present invention relates generally to the field of tooth preparation or reduction to receive a crown, and more particularly, to any apparatus and method for digitally recording motion of a handpiece-driven rotary cutting tool in a dental handpiece. The invention relates specifically to digital dental systems and methods for recording and monitoring tooth surface reduction as such reduction is accomplished with the handpiece-driven rotary cutting tool in realtime. According to some embodiments, the digital dental system provides signals alerting the dentist to change one of the line-of-draw surface tapers when the line-of-draw surface taper needs to be increased or decreased. According to some embodiments, the digital dental system provides other signals alerting the dentist to move on to other tooth surfaces when the tooth surface has been adequately reduced.

### BACKGROUND

Dental handpieces and cutting tools are well known and used in both practice and laboratory settings. Handpieces are designed to attach various sizes and shapes of cutting tools that are used for reducing the surfaces of teeth and treatment devices. To use a handpiece, a dentist or a technician must acquire knowledge and skills related to dental anatomy, fitting, and functioning contours.

Dentists are said to “prepare” a tooth when they use handpieces with handpiece-driven rotary cutting tools to reduce tooth surfaces to receive a crown. Handpieces have chucks or collets for attaching or holding cutting tools. Handpieces are precision high-speed pneumatic instruments used at speeds of 400,000 rpm (revolutions per minute) or more and, on contact, operate at speeds of 180,000-350,000 rpm. Skills required for using a handpiece include line-of-draw reduction and amount of reduction considerations.

Handpieces and attached cutting tools in general use today, do not facilitate digitizing in realtime, and accordingly do not monitor the motion of the cutting tool as the tooth surfaces are being reduced. Furthermore, handpieces and attached cutting tools have not been related to intelligent digital dental systems, so the dentist does not get any assistance in contouring the tooth surfaces as the surfaces are being reduced. Furthermore, the resulting reduction tooth is not stored in a digital file, so some other copying means, such as an impressioning technique or an independent digitizing device, must be used to relate the reduction tooth to the fabricated custom crown.

### SUMMARY

Operation of state-of-the-art handpieces and cutting tools requires specialized knowledge and skills. Such handpieces and cutting tools are not capable of realtime motion-capture digitizing. Accordingly, handpieces and cutting tools in general use today, cannot produce a digital file having a polygon mesh model of the reduction tooth.

### ADVANTAGES

Accordingly, several advantages of one or more aspects are as follows: To provide a digital dental system comprising sensors and a handpiece attaching a digitizing cutting tool, the system having the capacity to facilitate preparing of a tooth to generate a digital file. To provide the digital dental system having the capacity to record tooth surface data as it is created in realtime. To provide the digital dental system having the capacity to produce signals, alerting the dentist to increase or decrease one of the line-of-draw surface tapers as needed to fit a crown. To provide the digital dental system having the capacity to produce signals, alerting the dentist to move on to other tooth surfaces as the present tooth surface has been adequately reduced to fit a crown. To provide the digital dental system having the capacity to produce a digital file having a polygon mesh model of the tooth as it has been reduced. To provide the digital dental system having the capacity to facilitate preparing of a tooth that fits a premanufactured crown. To provide the digital dental system having the capacity to produce a deliverable digital file of a reduction tooth for facilitating fabrication of a custom crown.

### BRIEF DESCRIPTION OF THE DRAWINGS

The foregoing disclosure will be best understood and advantages thereof made most clearly apparent when consideration is given to the following detailed description in combination with the drawing figures presented. The detailed description makes reference to the following drawing figures:

FIG. 1 is an open mouth view of the intraoral input devices showing the sensor that is an integrated part of the handpiece and the sensor adhered to two teeth for digitally recording tooth surface data as it is created by the handpiece and attached cutting tool, according to some embodiments.

FIG. 2 is a diagrammatic illustration of the reduction tooth showing the unreduced surfaces, the minimally reduced surfaces, and the cutting tool, according to some embodiments.

FIG. 3 is a diagrammatic illustration of the reduction tooth showing the unreduced surfaces, the reduced surfaces, the line-of-draw surface tapers, and the cutting tool, according to some embodiments.

FIG. 4 is a diagrammatic illustration of the reduction tooth showing the unreduced surfaces, the reduced surfaces, the line-of-draw surface tapers, and the digitizing probe, according to some embodiments.

FIG. 5A is a flowchart showing certain steps in a method of preparing of a tooth to generate a digital file, according to some embodiments.

FIG. 5B is a flowchart showing certain additional steps in a method of preparing of a tooth to generate a digital file, according to some embodiments.

FIG. 6 is a diagrammatic illustration of a digital dental system showing technology-specific hardware and software, a computer, a polygon mesh model of the tooth being reduced, sensors, and a handpiece, according to some embodiments.

### TERMS ACCORDING TO THE SOME EMBODIMENTS

Arch, according to dental definition, is a structure comprising the teeth and the alveolar ridge. The upper arch is known as the maxillary arch and the lower arch is known as the mandibular arch. The arch of the tooth being prepared or reduced is part of the stable data or motion-negating data recorded during the tooth surface reduction digitizing procedure. That is, although a patient may move their head or jaw during the reduction digitizing procedures, these motions are related by a computer, to the motions of the handpiece-driven rotary cutting tool so that only cutting tool motions are recorded.

Computerized system, according to some embodiments, comprises; technology-specific hardware and software; and a computer having a processor, memory, graphics adapter and software.

Crown, according to dental definition, is a conventional restorative treatment for replacing missing or decayed parts of a tooth. A patient's tooth must be reduced to receive the crown. That is, the tooth surfaces must be reduced to constitute line-of-draw surfaces for inserting and cementing the crown. The crown may be a premanufactured crown, or it may be a custom fabricated crown.

Cutting tool, according to some embodiments, is a surface reducing device that is part of a digital dental system. According to some embodiments, the cutting tool may be a round-tip diamond coated dental bur, having the capacity to facilitate reduction of a tooth surface as the digital dental system records the reduced surfaces in real-time. The cutting tool is a device having dimensions that are known in a software program, and accordingly, the cutting tool functions as a digitizing probe. In some embodiments, the cutting tool may be detached from a handpiece and a digitizing probe may be attached to the handpiece to generate a plurality of margins

Digital data is data that represents forms of information using specific machine language systems that can be interpreted by various technologies.

Digital dental system, according to some embodiments, comprises; a handpiece having at least one integrated sensor and an attached cutting tool; at least one of the sensors adhered to the arch on which the tooth to be reduced is located; and a computerized system. According to some embodiments, the digital dental system is capable of recording motion of the cutting tool via the digital data from the sensors. Cooperation or cooperating connection between components of the digital dental system has the capacity of providing a plurality of wired or wireless connections.

Digital file, at a high level of abstraction, is a stored segment or block of information that is available to a computer program. According to some embodiments, a computerized system may store the digital file having a polygon mesh model of the tooth as it is being reduced in realtime. According to some embodiments, the computerized system may store the digital file having a polygon mesh model of the tooth as it has been reduced.

Digitizing probe, according to some embodiments, is a tool that is part of a digital dental system. The digitizing probe may be moved circumferentially against unreduced tooth surfaces, the reduced surface and the unreduced surface intersections producing an edge representing at least one margin in the digital file. According to some embodiments, the digitizing probe may be attached to the handpiece. According to some embodiments, the digitizing probe may be a separate component integrating at least one of the sensors, the digitizing probe and the sensor being part of the digital dental system.

Handpiece, according to dental definition, is an instrument used to hold a rotary cutting tool. The handpiece may be a pneumatic contra-angle instrument commonly used intraorally, to prepare or reduce a tooth to receive a crown. The handpiece may be part of a digital dental system. According to some embodiments, the handpiece may have one integrated sensor and an attached cutting tool that functions as a digitizing probe as the handpiece facilitates reduction of the tooth surfaces. According to some embodiments, the handpiece may have more than one integrated sensor and an attached cutting tool that functions as a digitizing probe as the handpiece facilitates reduction of the tooth surfaces. According to some embodiments, the handpiece may be an integrated part of an articulated arm and an attached cutting tool that functions as a digitizing probe as the handpiece facilitates reduction of the tooth surfaces. According to some embodiments, the handpiece, the cutting tool, and the sensors have the capacity to cooperate with the computerized system to record motion of the cutting tool, said motion generating the digital file of a plurality of the reduced surfaces.

Line-of-draw, according to dental definition, is the direction of insertion and withdrawal of a fixable or cementable restoration. The line-of-draw incorporates sides of a tooth that are tapered to some degree, such as 2-degrees.

Margin, according to dental definition, is a boundary line formed by a reduced surface and an unreduced surface of a tooth. According to some embodiments, a digitizing probe is attached on the handpiece and may be moved circumferentially against the unreduced tooth surfaces to generate at least one margin in the digital file. According to some embodiments, the digitizing probe may be a separate component integrating at least one of the sensors, the digitizing probe and the sensor being part of the digital dental system.

Reduction tooth, according to some embodiments, may be a tooth that is to be reduced, that is being reduced, or that has been reduced.

Reduction tooth library, according to some embodiments, is a digital collection of various sizes of molar, bicuspid, cuspid, and incisal teeth that have been prepared or reduced to receive crowns. According to some embodiments, the reduction tooth library may represent fitting surfaces of premanufactured crowns.

Sensor is a motion-tracking device that detects and responds to some type of input from the physical environment. Sensor technologies include optoelectronic cone beam, confocal laser, electro-magnetic, and ultrasound motion tracking systems. Each sensor technology may require technology-specific hardware and software. According to some embodiments, the specific sensor input may be motion, pressure, or vibration, or some combination thereof. According to some embodiments, the sensors, the handpiece, and the cutting tool, are elements of a digital dental system which is capable of generating a digital file having a polygon mesh model of the tooth as it is being reduced in realtime. According to some other embodiments, the digital dental system is also capable of generating a digital file having polygon mesh models of a group of the teeth in one of the arches, as the tooth surfaces are being reduced. The sensors track motion and digitize tooth surfaces being reduced by a cutting tool in realtime. According to some embodiments, the sensors may be located intraorally, extraorally, or a combination thereof. The sensor may be adhered to at least one tooth on the arch of the tooth to be reduced. According to some embodiments, other sensors may be adhered or provisionally attached to other teeth in the same arch. According to some other embodiments, sensors may be an integrated part of an impression material adhered extender, reaching out extraorally. According to some embodiments, sensors may be an integrated part of the handpiece handle, or an extended handle, reaching out, extraorally.

Tooth, according to some embodiments, is each of a set of hard, bony enamel-coated structures in the jaws of humans, used for chewing.

Tooth surfaces, according to some embodiments, comprise the designated sides of a tooth known as the facial, lingual, mesial, distal, and occlusal surfaces.

### DRAWING REFERENCE NUMERALS

100 upper lip
102 lower lip
104 cutting tool
106 handpiece
107 collet
108 tooth
110 sensor
111 provisional adhesive
112 arch
114 unreduced surface
116 reduced surface
118 line-of-draw surface taper
120 margin
122 digitizing probe
124 step “Enter tooth to be reduced into digital dental system.”
126 step “Provisionally adhere at least one sensor to arch on which tooth to be reduced is located.”
128 step “Attach cutting tool on handpiece, said handpiece having at least one integrated sensor.”
130 step “Move cutting tool minimally against plurality of facial, lingual, mesial, distal, and occlusal surfaces of tooth being reduced.”
132 step “Access digital file of minimally reduced reduction tooth to facilitate selection of reduction tooth size from reduction tooth library.”
134 step “Reduce surfaces of reduction tooth until digital dental system respectively signals to increase or decrease line-of-draw surface tapers.”
136 step “Reduce surfaces of reduction tooth until digital dental system respectively signals to move on to other surfaces of reduction tooth.”
138 step “Move digitizing probe circumferentially against unreduced surfaces of reduction tooth generating margins in digital file.”
140 step “Access digital file of reduction tooth to facilitate selection of premanufactured crown that fits reduction tooth.”
142 step “Access digital file of reduction tooth to facilitate production of physical or virtual model of reduction tooth.”
144 step “Access digital file of reduction tooth to facilitate production of custom crown that fits reduction tooth.”
146 computer
148 technology-specific hardware and software
150 cooperating connection
152 pneumatic connection
154 polygon mesh model
1000 intraoral input devices
2000 reduction tooth
3000 digital dental system

### DETAILED DESCRIPTION OF EXEMPLARY EMBODIMENTS

In the following description of exemplary embodiments, reference is made to the accompanying drawings which form a part hereof, and in which it is shown by way of illustration, specific exemplary embodiments can be practiced. It is to be understood that other embodiments can be used and structural changes can be made without departing from the spirit and scope.

Disclosed herein is a class of motion tracking apparatus which uses sensors and a cutting tool to record tooth surface reduction in realtime. At least one sensor on a dental handpiece tracks the motion of the cutting tool and at least one sensor adhered to teeth in the same arch tracks nonmotion of the arch. The embodiments have the benefits of a digital dental system for configuring a reduction tooth to generate a digital file.

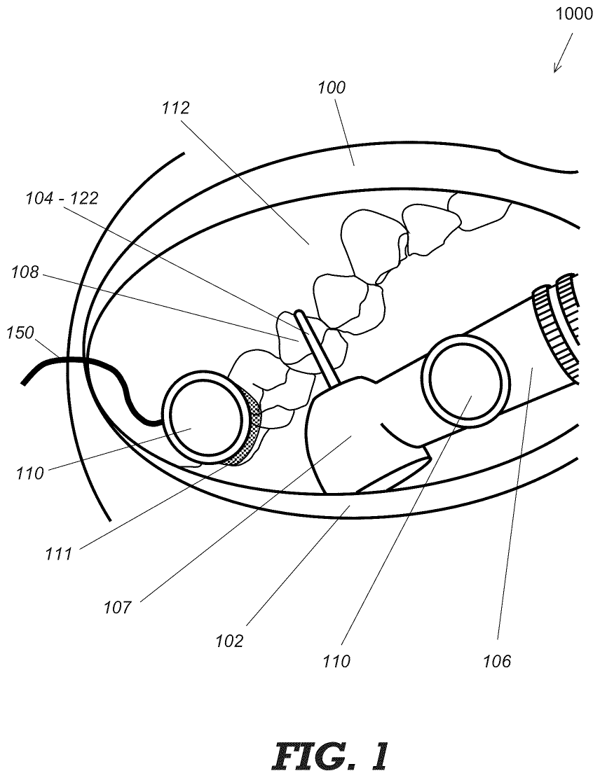

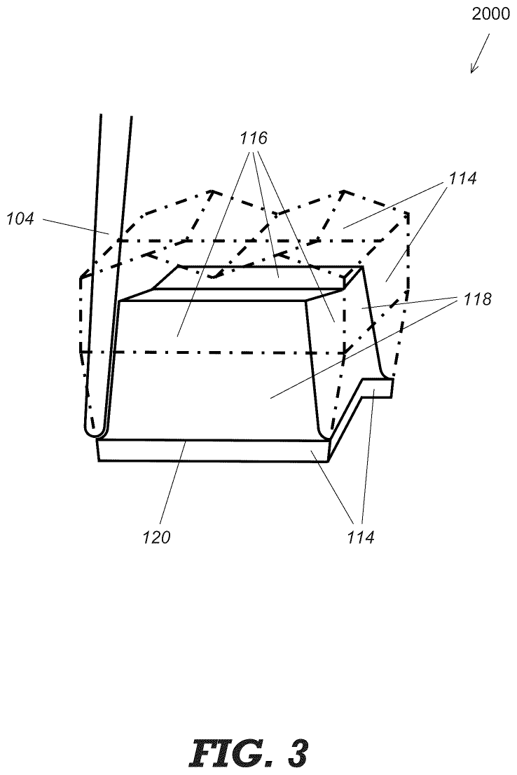

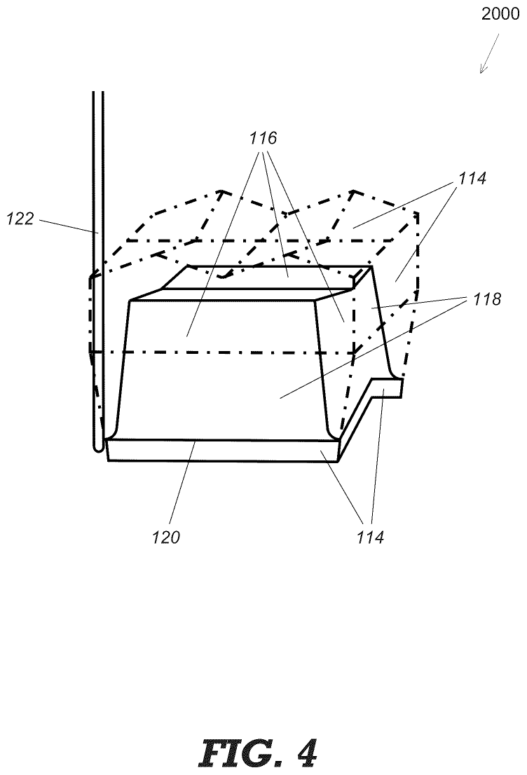

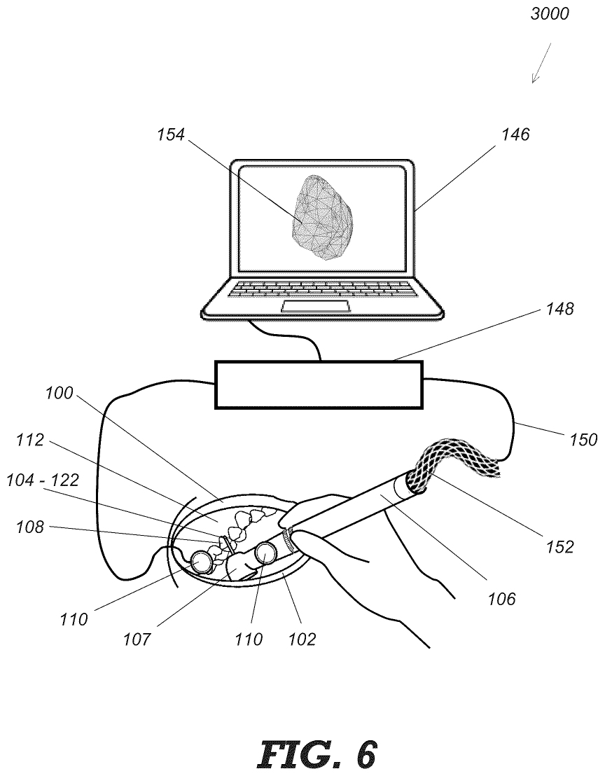

FIG. 1 is an open mouth view of a plurality of intraoral input devices 1000 showing an upper lip 100 and a lower lip 102. In this illustration, a cutting tool 104 or a digitizing probe 122 may be attached to a handpiece 106 via a collet 107. In some embodiments, the cutting tool 104 attached to the handpiece 106 may be used to prepare or reduce a tooth 108. In some embodiments, the digitizing probe 122 attached to the handpiece 106 may be used to generate at least one margin 120, shown in FIGS. 3 and 4. At least one sensor 110 is shown which is an integrated part of the handpiece 106. At least one other of the sensors 110 is shown which has been adhered, using a provisional adhesive 111, to two teeth 108 on an arch 112 of the tooth 108 to be reduced. The cutting tool 104, the handpiece 106, and the sensors 110 are elements of a digital dental system 3000, shown in FIG. 6, for generating a digital file having a polygon mesh model 154, shown in FIG. 6, of the tooth 108 as it is being reduced in realtime. The digital dental system 3000 comprises; a computerized system; a plurality of cooperating connections 150; and the handpiece 102 attaching a cutting tool 104 or a digitizing probe 122. The computerized system comprises; a plurality of technology-specific hardware and software 148, shown in FIG. 6; and a computer 146, shown in FIG. 6, having a processor, memory, graphics adapter, and software for recording motion of the cutting tool 104 via the digital data from the sensors 110. According to some embodiments, the digital dental system 3000 is also capable of generating the digital file of a group of the teeth 108 on the arch 112 as the teeth 108 are being reduced in realtime. According to some embodiments, the sensors 110 may be located intraorally, extraorally, or a combination thereof. According to some embodiments, the sensors 110 may be an integrated part of the handpiece 106 handle, or an extended handle, reaching out extraorally. According to some embodiments, other sensors 110 may be adhered to other teeth 108 in the same arch 112, and the sensors 110 may be part of the arch 112 adhered extender reaching out, extraorally. According to some embodiments, the digital dental system 3000 is also capable of generating the digital file of the tooth 108 as it has been prepared. The digital file may facilitate selection of the premanufactured crown that fits the tooth 108 as it has been prepared. The digital file may facilitate production of a physical or virtual model of the tooth 108 as it has been prepared. The digital file also may facilitate a custom crown that fits the tooth 108 as it has been prepared.

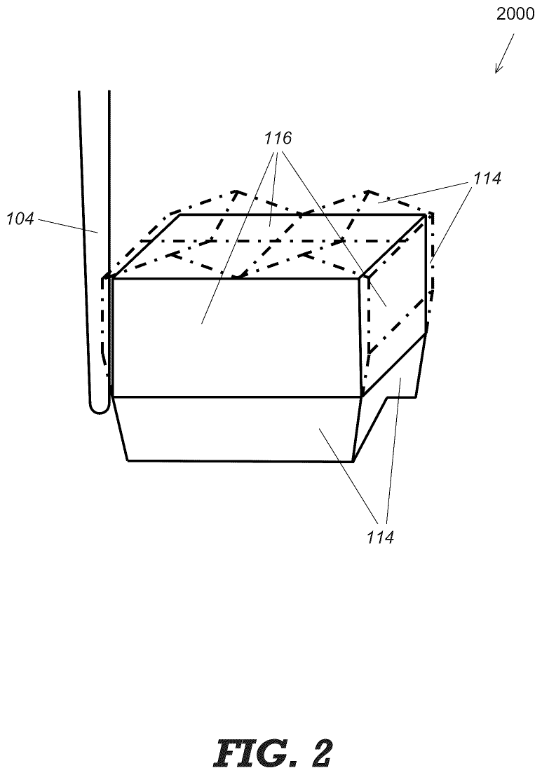

FIG. 2 is a diagrammatic illustration of a reduction tooth 2000 showing a plurality of unreduced surfaces 114 and a plurality of reduced surfaces 116, the reduced surfaces 116 having minimal surface reduction. In this illustration, the cutting tool 104 is being used to initiate reduction of the reduction tooth 2000 surfaces. This reduction of the surfaces of the reduction tooth 2000 comprises minimal reduction of a plurality of facial, lingual, mesial, distal, and occlusal surfaces to acquire a digital data set for selection of the reduction tooth 2000 size from a reduction tooth library.

FIG. 3 is a diagrammatic illustration of the reduction tooth 2000 showing the unreduced surfaces 114, the reduced surfaces 116, and a plurality of line-of-draw surface tapers 118. In this illustration, the cutting tool 104 is being used to reduce the surfaces of the reduction tooth 2000 thereby producing at least one of the margins 120. According to some embodiments, reduction of the surfaces of the reduction tooth 2000 continues with monitored reduction of each surface in turn, each surface being respectively reduced until at least one auditory, lighted, or vibration signal generator alerts a dentist to increase or decrease one of the line-of-draw surface tapers 118. According to some embodiments, reduction of the surfaces of the reduction tooth 2000 continues with monitored reduction of each surface in turn, each surface being respectively reduced until the signal generator alerts the dentist to move on to another surface, as the present surface has been sufficiently reduced.

FIG. 4 is a diagrammatic illustration of the reduction tooth 2000 showing the unreduced surfaces 114, the reduced surfaces 116, and the line-of-draw surface tapers 118. In this illustration, the digitizing probe 122 is being used to locate and generate at least one of the margins 120 in the digital file. Subsequent to reduction of the surfaces of the reduction tooth 2000, the cutting tool 104 may be detached from the handpiece 106, the digitizing probe 122 being attached in its place. The digitizing probe 122 may then be moved circumferentially against the unreduced surfaces 114 of the reduction tooth 2000, the reduced surface 116 and the unreduced surface 114 intersections generating at least one of the margins 120 in the digital file. According to some embodiments, the digitizing probe 122 may be a separate component integrating at least one of the sensors 110, the digitizing probe 122 and the sensor 110 being part of the digital dental system 3000, shown in FIG. 6. The handpiece 106, the cutting tool 104, and the sensors 110 have the capacity to cooperate with the digital dental system 3000 to generate the digital file having the polygon mesh model of the reduction tooth 2000. The digital file may facilitate selection of the premanufactured crown that fits the reduction tooth 2000. The digital file also may facilitate production of the physical or virtual model of the arch 112 incorporating the model of the reduction tooth 2000. The digital file also may facilitate production of a custom crown that fits the reduction tooth 2000.

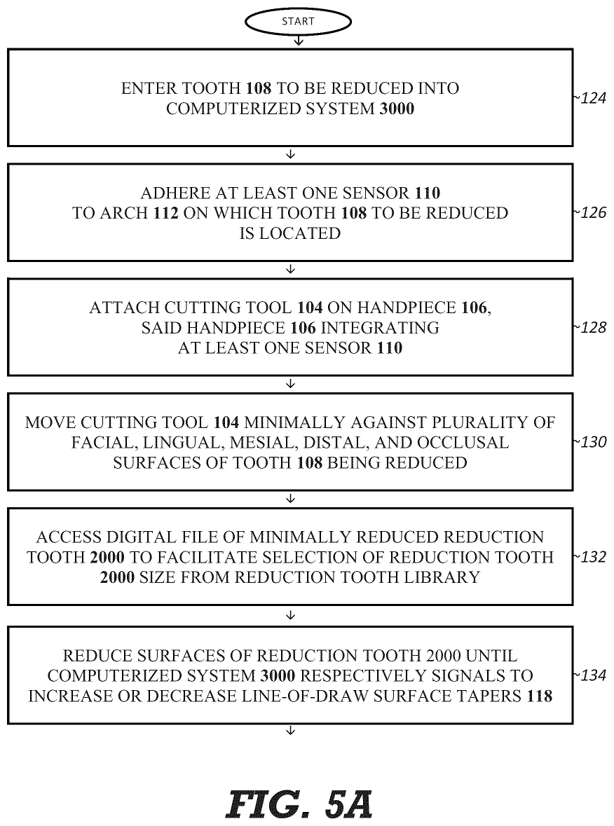

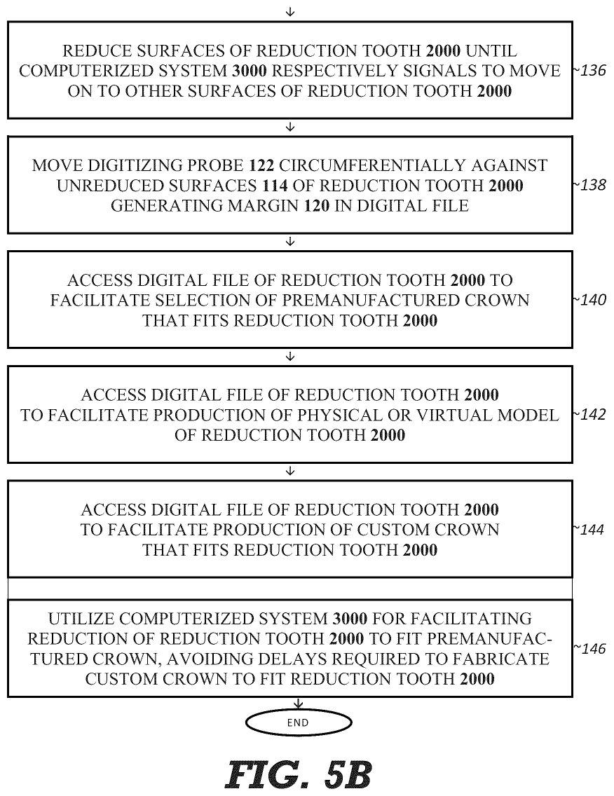

FIGS. 5A and 5B are a flowchart showing certain steps 124-144 in a method of configuring the reduction tooth 2000 to generate a digital file according to the present disclosure. A plurality of computer data signals being applied in the present disclosure are reproducible computer-readable signals embodied in carrier waves. At step 124 the tooth 108 being reduced may be entered into the digital dental system 3000, shown in FIG. 6. At step 126 at least one of the sensors 110 may be provisionally adhered to the arch 112 on which the tooth 108 to be reduced may be located. At step 128 the cutting tool 104 may be attached on the handpiece 106. the handpiece 106 integrating at least one of the sensors 110. At step 130 the cutting tool 104 may be minimally moved against the plurality of facial, lingual, mesial, distal, and occlusal surfaces of the reduction tooth 2000, thereby reducing the reduction tooth 2000 surfaces as little as possible. At step 132 the digital file of the minimally reduced reduction tooth 2000 may be accessed to facilitate selection of the reduction tooth 2000 size from the reduction tooth library. At step 134 surfaces of the reduction tooth 2000 may be reduced until the digital dental system 3000 respectively signals to increase or decrease one of the line-of-draw surface tapers 118. At step 136 surfaces of the reduction tooth 2000 may be reduced until the digital dental system 3000 respectively signals to move on to other surfaces of the reduction tooth 2000. At step 138 the digitizing probe 122 may be moved circumferentially against the unreduced surfaces 114 of the reduction tooth 2000 generating at least one of the margins 120 in the digital file. At step 140 the digital file of the reduction tooth 2000 may be accessed to facilitate specification of the premanufactured crown that fits the reduction tooth 2000. At step 142 the digital file of the reduction tooth 2000 may be accessed to facilitate production of the physical or virtual model of the reduction tooth 2000. At step 144 the digital file of the reduction tooth 2000 may be accessed to facilitate production of the custom crown that fits the reduction tooth 2000.

FIG. 6 is a diagrammatic illustration of the digital dental system 3000 showing a computer 146, the technology-specific hardware and software 148, a plurality of cooperating connections 150, and the handpiece 102 comprising a pneumatic connection 152. Also shown is an open mouth of a patient including the upper lip 100 and the lower lip 102. In this illustration, the cutting tool 104 is attached to the handpiece 106 via a collet 107. The handpiece 106 and the cutting tool 104 are being used to reduce the tooth 108. At least one of the sensors 110 is shown which is an integrated part of the handpiece 106. At least one other of the sensors 110 is shown which has been adhered to at least one tooth 108 on the arch 112 of the tooth 108 to be reduced. The cutting tool 104, the pneumatic handpiece 106, and the sensors 110 are elements of a digital dental system 3000 for generating a digital file including the polygon mesh model 154 of the reduction tooth 2000 as the reduction tooth 2000 is being reduced in realtime. According to some embodiments, the handpiece 106, the cutting tool 104, and the sensors 110 having the capacity to cooperate with a computerized system to record motion of the cutting tool 104, said motion generating the digital file of a plurality of the reduced surfaces 116. The computerized system comprises, the technology-specific hardware and software 148, and the computer 146 having a processor, memory, graphics adapter, and software for recording motion of the cutting tool 104 via the digital data from the sensors 110. According to some embodiments, the digital dental system 3000 is also capable of generating the digital file of a group of the teeth 108 on the arch 112 as the teeth 108 are being reduced in realtime. According to some embodiments, the sensors 110 may be located intraorally, extraorally, or a combination thereof. According to some embodiments, the sensors 110 may be an integrated part of the handpiece 106 handle, or an extended handle, reaching out extraorally. According to some embodiments, other sensors 110 may be adhered to other teeth 108 in the same arch 112. According to some embodiments, the sensors 110 may be an integrated part of the arch 112 adhered extender reaching out, extraorally. According to some embodiments, the digital dental system 3000 is also capable of generating the digital file of the tooth 108 as it has been reduced. The digital file may facilitate selection of the premanufactured crown that fits the tooth 108 as it has been reduced. The digital file may facilitate production of a physical or virtual model of the tooth 108 as it has been reduced. The digital file also may facilitate production of a custom crown that fits the tooth 108 as it has been reduced.

### CONCLUSIONS, RAMIFICATIONS, AND SCOPE

Accordingly, the reader will see that the digital dental system comprising the sensors and the handpiece attaching the digitizing cutting tool has the capacity to facilitate configuring of the reduction tooth to generate the digital file; that the digital dental system has the capacity to record reduction tooth surface data as it is created in realtime; that the digital dental system has the capacity to produce signals, alerting the dentist to increase or decrease one of the line-of-draw surface tapers as needed to fit a crown; that the digital dental system has the capacity to produce signals, alerting the dentist to move on to other reduction tooth surfaces as the present reduction tooth surface has been adequately reduced to fit the crown; that the digital dental system has the capacity to produce the digital file having the polygon mesh model of the reduction tooth as it has been configured; that the digital dental system has the capacity to facilitate preparing of the reduction tooth that fits the premanufactured crown; that the digital dental system has the capacity to produce the deliverable digital file of the reduction tooth for facilitating fabrication of the custom crown.

Although the description above contains many specifications, these should not be construed as limiting the scope of the embodiments but as merely providing illustrations of some of several embodiments. For example; the sensors may be located intraorally, extraorally, or a combination thereof; the sensors may be an integrated part of an impression material adhered extender, reaching out extraorally; the sensors may be an integrated part of the handpiece handle, or an extended handle, reaching out extraorally; the cutting tool, the handpiece, and the sensors may be elements of a digital dental system for generating a digital file of a group of the teeth as they are being reduced in realtime; the digitizing probe may be a separate component integrating at least one of the sensors, the digitizing probe and the sensor being part of the digital dental system; the dentist may choose to permanently cement a premanufactured crown; the dentist may choose to temporarily cement the premanufactured crown and subsequently, to permanently cement a custom crown, etc.

While various embodiments have been described above, it should be understood that they have been presented by way of example only, and not by way of limitation. Likewise, the various illustrations may depict an example design or other configuration for the disclosure, which illustrations are done to aid in understanding the features and functionality that can be included in the disclosure. The disclosure is not restricted to the illustrated examples, designs, or configurations, but can be implemented using a variety of alternative designs and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. Thus, the breadth and scope of the present disclosure should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; and adjectives such as “conventional,” “traditional,” “normal,” “standard,” “known,” and terms of similar meaning, should not be construed as limiting the item described to a given time period, or to an item available as of a given time. But instead, these terms should be read to encompass conventional, traditional, normal, or standard technologies that may be available, known now, or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to,” or other like phrases in some instances shall not be read to mean that the narrower case is intended or required in instances where such broadening phrases may be absent.

1. A computerized system (3000) for preparing or reducing at least one tooth (108) and generating a digital file, the computerized system (3000) comprising:
a) at least one sensor (110) having the capacity to be adhered to an arch (112) or at least one of the teeth (108) on the arch (112) on which the tooth (108) to be reduced is located;
b) at least one of the sensors (110) being an integrated part of a handpiece (106), the handpiece (106) having the capacity to attach a cutting tool (104); and
c) further comprising the handpiece (106) and the cutting tool (104) having the capacity to cooperate with the sensors (110) and other components of the computerized system(3000) to record motion of the cutting tool (104), said motion generating the digital file of a plurality of the reduced surfaces (116) in realtime, the digital file facilitating production of a physical or virtual model of a reduction tooth (2000) and a custom crown that fits the reduction tooth (2000).

2. The system of claim 1, wherein the handpiece (106) has the capacity to detach the cutting tool (104) and to attach a digitizing probe (122), the digitizing probe (122) having the capacity to be moved circumferentially against a plurality of unreduced surfaces (114) of the reduction tooth (2000), the reduced surface (116) and the unreduced surface (114) intersections data from the sensors (110) generating at least one margin (120) in the digital file.

3. The system of claim 1, wherein the computerized system (3000) further comprises a computer (146) having a processor, memory, graphics adapter, and sensor technology hardware and software (148) for recording motion of the cutting tool (104) via the digital data from the sensors (110).

4. The system of claim 1, wherein the computerized system (3000) has the capacity to record minimal cutback motion of the cutting tool (104), said motion facilitating specification of at least one of the reduction tooth (2000) sizes from a tooth preparation library.

5. The system of claim 1, wherein the computerized system (3000) has the capacity to facilitate monitored reduction of the reduction tooth (2000) in realtime, each surface of the reduction tooth (2000) being respectively reduced until a signal alerts a dentist to increase or decrease one of a plurality of line-of-draw surface tapers (118) and when to move on to other surfaces of the reduction tooth (2000).

6. The system of claim 5, wherein the digital file of the reduction tooth (2000) facilitates selection of a premanufactured crown that fits the reduction tooth (2000).

7. A method of using a computerized system (3000) for preparing or reducing at least one tooth (108) and generating a digital file, the method comprising the steps of:
a) adhering at least one sensor (110) to an arch (112) or at least one of the teeth (108) on the arch (112) on which the tooth (108) to be reduced is located;
b) reducing surfaces of a reduction tooth (2000) using a handpiece (106) and a cutting tool (104), the handpiece (106) having the capacity to attach the cutting tool (104) and the handpiece (106) integrating at least one of the sensors (110);
c) generating the digital file of a plurality of reduced surfaces (116) in realtime; and
d) accessing the digital file of the reduction tooth (2000) thereby facilitating production of a custom crown that fits the reduction tooth (2000).

8. The method of claim 7, further comprising the step of entering the tooth (108) being reduced into the computerized system (3000).

9. The method of claim 8, further comprising the step of moving the cutting tool (104) minimally against the surfaces of the reduction tooth (2000).

10. The method of claim 9, further comprising the step of accessing the digital file of the reduction tooth (2000) thereby facilitating specification of the reduction tooth (2000) size from a tooth preparation library.

11. The method of claim 10, further comprising the step of reducing surfaces of the reduction tooth (2000), each surface being reduced until the computerized system (3000) respectively signals to increase or decrease one of a plurality of line-of-draw surface tapers (118).

12. The method of claim 11, further comprising the step of reducing surfaces of the reduction tooth (2000), each surface being reduced until the computerized system (3000) respectively signals to move on to other surfaces of the reduction tooth (2000).

13. The method of claim 12, further comprising the step of moving a digitizing probe (122) circumferentially against a plurality of unreduced surfaces (114) of the reduction tooth (2000) thereby generating at least one margin (120) in the digital file.

14. The method of claim 13, further comprising the step of accessing the digital file of the reduction tooth (2000) thereby facilitating selection of a premanufactured crown that fits the reduction tooth (2000).

15. The method of claim 14, further comprising the step of accessing the digital file of the reduction tooth (2000) thereby facilitating production of a physical or virtual model of the reduction tooth (2000).

16. The system of claim 6, wherein the computerized system (3000) has the capacity to facilitate reduction of the reduction tooth (2000) to fit the premanufactured crown, thereby avoiding delays required to custom fabricate the crown to fit the reduction tooth (2000).

17. The method of claim 14, further comprising the step of utilizing the computerized system (3000) for facilitating reduction of the reduction tooth (2000) to fit the premanufactured crown, thereby avoiding delays required to fabricate the custom crown to fit the reduction tooth (2000).
